(19) United States
(12) Patent Application Publication
(10) Pub. No.: US 2007/0208088 A1
(43) Pub. Date: Sep. 6, 2007

(54) PQS: A NEW HYDROTROPE FOR SOLUBILIZING LIPOPHILIC COMPOUNDS IN WATER

(75) Inventor: Bruce H. Lipshutz, Goleta, CA (US)

Correspondence Address: MORGAN, LEWIS & BOCKIUS LLP (SF), 2 PALO ALTO SQUARE, 3000 El Camino Real, Suite 700, PALO ALTO, CA, 94306, US

(73) Assignee: ZYMES, LLC, Hasbrouck Heights, NJ (US)

(21) Appl. No.: 11/675,539

(22) Filed: Feb. 15, 2007

Related U.S. Application Data
(60) Provisional application No. 60/773,951, filed on Feb. 15, 2006.

(51) Int. Cl. A61K 47/00; C07F 5/02; C07D 207/46

(52) U.S. Cl. 514/772; 514/784; 548/571; 562/7; 562/43; 562/471

(57) ABSTRACT

The present invention provides a solubilizing agent of the general formula: 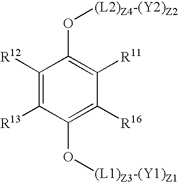
wherein R¹¹, R¹² and R¹³ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. L1 and L2 are linker moieties, which are members independently selected from substituted or unsubstituted alkyl and substituted or unsubstituted heteroalkyl. Y1 and Y2 are hydrophilic moieties, which are members independently selected from polyethers, polyalcohols and derivatives thereof. Z1, Z2, Z3 and Z4 are members independently selected from 0 and 1, wherein at least one of Z1 and Z2 is 1.

### CROSS-REFERENCES TO RELATED APPLICATIONS

The present application claims the benefit of U.S. Provisional Patent Application No. 60/773,951, filed Feb. 15, 2006, which is incorporated herein by reference in its entirety for all purposes.

### BACKGROUND OF THE INVENTION

Many bioactive compounds are highly lipophilic. While soluble in lipid formulations and some organic solvents, they are substantially insoluble or poorly soluble in aqueous media, which limits the use of those molecules in therapeutic applications. When administered as an oil solution, a suspension or an emulsion, lipophilic compounds are often characterized by low bioavailability and unfavorable pharmacokinetics. The lack of bioavailability is usually independent of the administration route (topical, oral, or parenteral).

Various approaches to overcome this limitation are known in the prior art. One approach consists of dissolving a lipophilic compound in a water-miscible organic solvent, such as ethanol or propylene glycol. However, when such a solution comes into contact with blood or gastrointestinal fluids, the lipophilic compound often precipitates as a solid or liquid emulsion, and as a result its bioavailability decreases. Furthermore, many lipophilic compounds are not soluble in water-miscible, organic solvents. In another approach, lipophilic compounds are part of multiphase emulsions containing oils and solvents in combination with surfactants. These compositions may improve the bioavailability, but do not significantly increase the solubility of a lipophilic compound in aqueous media, and are usually used in topical applications only. Therefore, these formulations are of little value for therapeutic uses where the preferred route of administration is oral or intravenous. Another technology uses vitamin E, or a sterol attached to hydrophilic moieties as a solubilizing agent for lipophilic compounds (U.S. Pat. No. 6,632,443 to Borowy-Borowski et al.). However, the indigestion of larger amounts of vitamin E, for instance, has been associated with safety concerns.

The present invention provides a new method of solubilizing hydrophobic compounds, which overcomes many prior art drawbacks and limitations.

### SUMMARY OF THE INVENTION

According to one aspect, the present invention provides solubilizing agents having a structure according to Formula (I):

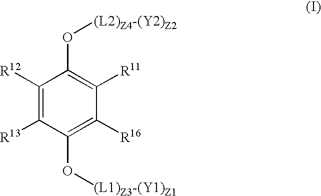

In Formula (I), R¹¹, R¹² and R¹³ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R¹² and R¹³, along with the atoms to which they are attached, are optionally joined to form a 4 to 8 membered ring. R¹⁶ is a member selected from OR¹⁷, SR¹⁷, NR¹⁷R¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R¹⁷ and R¹⁸ are members independently selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. L1 and L2 are linker moieties, which are members independently selected from substituted or unsubstituted alkyl and substituted or unsubstituted heteroalkyl. Y1 and Y2 are hydrophilic moieties, which are members independently selected from polyethers, polyalcohols and derivatives thereof. Z1, Z2, Z3 and Z4 are members independently selected from 0 and 1. This aspect has the proviso that at least one of Z1 and Z2 is 1, and with the further proviso that when Z4 and Z2 are both 0, (L2)_{z4}-(Y2)_{z2} is a member selected from H, a negative charge, and a salt counter ion, and when Z3 and Z1 are both 0, (L1)_{z3}-(Y1)_{z1} is a member selected from H, a negative charge, and a salt counter ion. In a preferred embodiment, Y1, Y2, L1 and L2 do not comprise a labeling moiety, a targeting moiety or a drug moiety.

According to another aspect, the present invention provides a water-soluble composition including the solubilizing agent according to Formula (I) and a bioactive lipophilic compound.

According to another aspect, the present invention provides a method of solubilizing a bioactive lipophilic compound in a water-based liquid, comprising contacting said bioactive lipophilic compound and a hydrophilic solvent (e.g., a water-based liquid) in the presence of a solubilizing agent according to Formula (I). In one embodiment, the hydrophilic solvent includes the solubilizing agent.

According to another aspect, the present invention provides a method of performing a reaction between a bioactive lipophilic compound and a hydrophilic reactant, comprising contacting the hydrophilic reactant and a water-soluble composition of the invention, wherein the water-soluble composition includes a solubilizing agent according to Formula (I) and the bioactive lipophilic compound.

According to still another aspect, the invention provides pharmaceutical or cosmetic formulations comprising the solubilizing agent according to Formula (I).

According to yet another aspect, the invention provides novel solubilizing agents of Formula (I) and methods of their preparation and purification.

### DETAILED DESCRIPTION OF THE PREFERRED EMBODIMENTS

I. Definitions and Abbreviations

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization are those well known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, -CH₂—CH₂—S(O)₂—CH₃, —CH═CH—O—CH₃, —Si(CH₃)₃, —CH₂—CH═N—OCH₃, and —CH═CH—N(CH₃)—CH₃, Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′—represents both —C(O)₂R′— and —R′C(O)₂—.

In general, an “acyl substituent” is also selected from the group set forth above. As used herein, the term “acyl subsituent” refers to groups attached to, and fulfilling the valence of a carbonyl carbon that is either directly or indirectly attached to the polycyclic nucleus of the compounds of the present invention.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quatemized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazol 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroakyl substituents,” respectively, and they can be one or more of a variety of groups selected from, but not limited to: —OR′, ═O, ═NR′, ═N—OR′, —NR′R″, —SR′, -halogen, -SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR′″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R′″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R′″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, the aryl substituents and heteroaryl substituents are generally referred to as “aryl substituents” and “heteroaryl substituents,” respectively and are varied and selected from, for example: halogen, —OR′, ═O, ═NR′, ═N—OR′, —NR′R″, —SR″, -halogen, -SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R′″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R′″ groups when more than one of these groups is present.

Two of the aryl substituents on adjacent atoms of the aryl or heteroaryl ring may optionally be replaced with a substituent of the formula -T—C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂), —B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula—(CRR′)s—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S), phosphorus (P) and silicon (Si).

The term “labeling moiety” refers to a moiety, which provides a signal that is detectable by a detection method known in the art. The signal can be used to determine the location or concentration of the labeling moiety, for example, in an organism, a tissue sample or a reaction vial. Exemplary signals include color, emitted light of any wavelength, radioactivity, or any other electromagnetic or quantum mechanical effect. Exemplary labeling moieties include but are not limited to fluorescent molecules (e.g. fluorescein), luminescent moieties (e.g., transition-metal complexes), chemoluminescent molecules, molecules used in colorimetric applications (i.e. dye molecules), histochemical staining reagents, photoaffinity labels, magnetic resonance imaging (MRI) agents, radioactive labels, radiotracers and agents used in positron emission tomography (PET).

The term “targeting moiety” refers to a moiety which is capable of binding to a particular tissue- or cell-type (e.g., tumor cells, neuronal or glial cells, liver cells, and the like) with at least some level of specificity. Exemplary targeting moieties are selected from carbohydrates, proteins, peptides, antibodies, and small-molecule ligands. In an exemplary embodiment, the targeting moiety is a ligand for a biological receptor, such as a cell surface receptor. In another exemplary embodiment, the targeting moiety is an antibody that is capable of binding to an antigen, such as a tissue- or tumor-specific antigen.

The term “drug moiety” refers to pharmaceutical drugs and other biologically active molecules. “Drug moiety” includes small-molecule drugs as well as biologics, including peptides, mutant and wild-type polypeptides, mutant and wild-type proteins, antibodies (e.g., humanized, monoclonal antibodies) and the like.

The term “water-soluble” refers to moieties that have a detectable degree of solubility in water. Methods to detect and/or quantify water solubility are well known in the art. Exemplary water-soluble polymers include peptides, saccharides, poly(ethers), poly(amines), poly(carboxylic acids) and the like. Peptides can have mixed sequences of be composed of a single amino acid, e.g., poly(lysine), poly(aspartic acid), and poly(glutamic acid). An exemplary polysaccharide is poly(sialic acid). An exemplary poly(ether) is poly(ethylene glycol), e.g., m-PEG. Poly(ethylene imine) is an exemplary polyamine, and poly(acrylic) acid is a representative poly(carboxylic acid).

As used herein, “pharmaceutically acceptable carrier” includes any material, which when combined with the conjugate retains the conjugates' activity and is non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

As used herein, “administering” means oral administration, administration as a suppository, topical contact, intravenous, intraperitoneal, intramuscular, intralesional, or subcutaneous administration, administration by inhalation, or the implantation of a slow-release device, e.g., a mini-osmotic pump, to the subject. Administration is by any route including parenteral and transmucosal (e.g., oral, nasal, vaginal, rectal, or transdermal), particularly by inhalation. Parenteral administration includes, e.g., intravenous, intramuscular, intra-arteriole, intradermal, subcutaneous, intraperitoneal, intraventricular, and intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, transdermal patches, etc.

“Hydrotrope”, as used herein, refers to a class of water soluble organic molecules that significantly increase the aqueous solubility of certain lipophilic compounds. Hydrotropes are similar to surfactants but typically possess a smaller hydrophobic moiety. The reduced hydrophobic moiety makes hydrotropes less prone to forming micelles. An example of a hydrotrope is sodium xylenesulfonate, which is used in the consumer product industry. Other examples include nicotinamide, sodium ascorbate, cyclodextrins, liposomes and nanoparticles.

II. The compositions

According to one aspect, the present invention provides a solubilizing agent as described herein. In an exemplary embodiment, the solubilizing agent has a structure according to Formula (I):

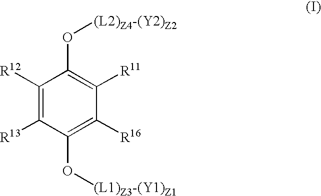

In Formula (I), R¹¹, R¹² and R¹³ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R¹⁶ is a member selected from OR¹⁷, SR¹⁷, NR¹⁷R¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R¹⁷ and R¹⁸ are members independently selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R¹² and R¹³, along with the atoms to which they are attached, are optionally joined to form a 4- to 8-membered ring. L1 and L2 are linker moieties, which are members independently selected from substituted or unsubstituted alkyl and substituted or unsubstituted heteroalkyl.

Y1 and Y2 are polymeric hydrophilic moieties, which are members independently selected from polyethers, polyalcohols and derivatives thereof. In a preferred embodiment, Y1, Y2, L1 and L2 do not comprise a labeling moiety, a targeting moiety or a drug moiety. Z1, Z2, Z3 and Z4 are members independently selected from 0 and 1 with the proviso that at least one of Z1 and Z2 is 1. When Z4 and Z2 are both 0, (L2)_{z4}-(Y2)_{z2} is preferably a member selected from H, a negative charge, and a salt counterion, and when Z3 and Z1 are both 0, (L1)_{z3}-(Y1)_{z1} is preferably a member selected from H, a negative charge, and a salt counterion.

The hydrophobic moiety of the solubilizing agent is a hydrophobic molecule having an esterifiable hydroxy group and is preferably a ubiquinol or a derivative thereof. Ubiquinol is a reduced derivative of a ubiquinone.

In an exemplary embodiment, R¹⁵ includes a structure according to the following formula: 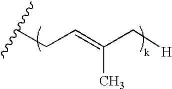
wherein k is an integer of from 1 to 13.

Preferred ubiquinols are those of Formula (II): 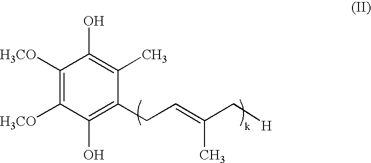
wherein k is an integer of from 1 to 13. The ubiquinol with k equal to 10 (ubiquinol-50 or reduced coenzyme Q₁₀) is particularly preferred.

In an exemplary embodiment R¹¹ in Formula (I) is H. Exemplary ubiquinol analogs according to this aspect of the invention include:

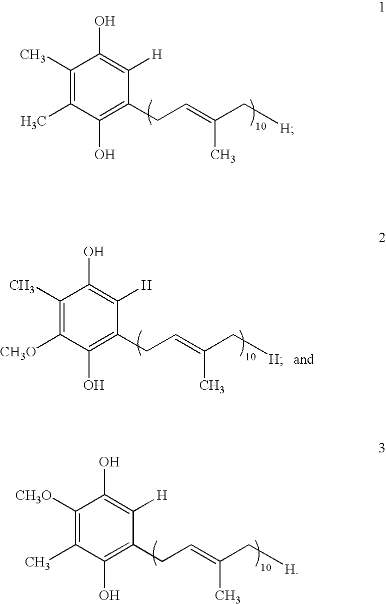

In another embodiment of the invention R¹¹ is a methyl group and R¹² and R¹³ are members independently selected from H, unsubstituted alkyl, unsubstituted alkoxy, halogen substituted alkyl, and halogen substituted alkoxy.

Exemplary compounds according to this aspect of the invention include:

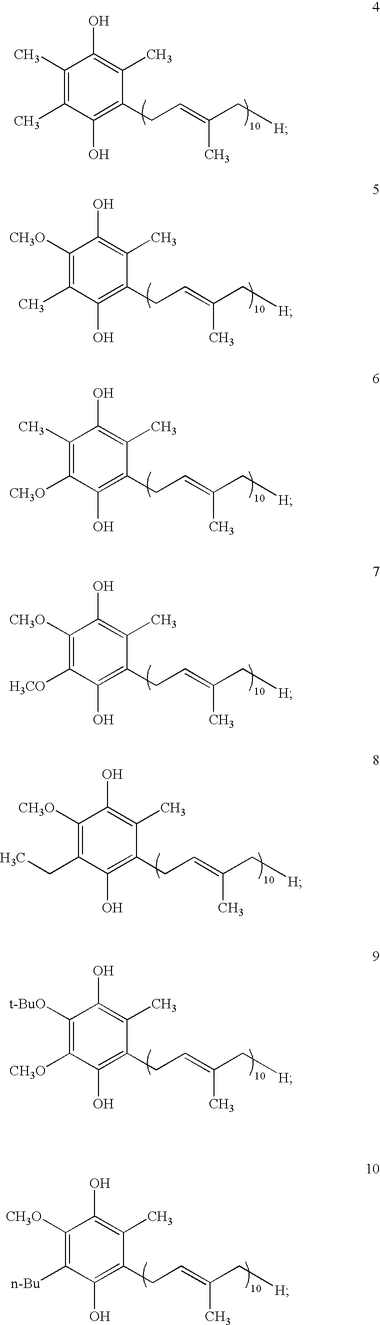

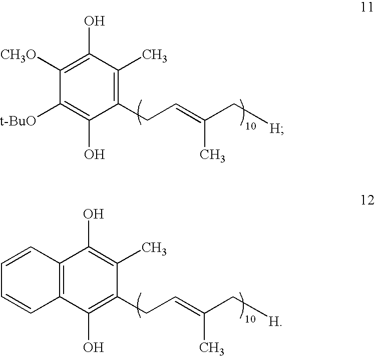

In another exemplary embodiment, one or more of the substituents R¹¹, R¹² and R¹³ include halogen atoms. In another exemplary embodiment the halogen is fluoro. Exemplary fluoroalkyl and fluoroalkoxy groups according to this aspect of the invention include but are not limited to CF₃, OCF₃, CHF₂, OCHF₂, CH₂F, and OCH₂F.

Exemplary compounds according to this aspect of the invention include:

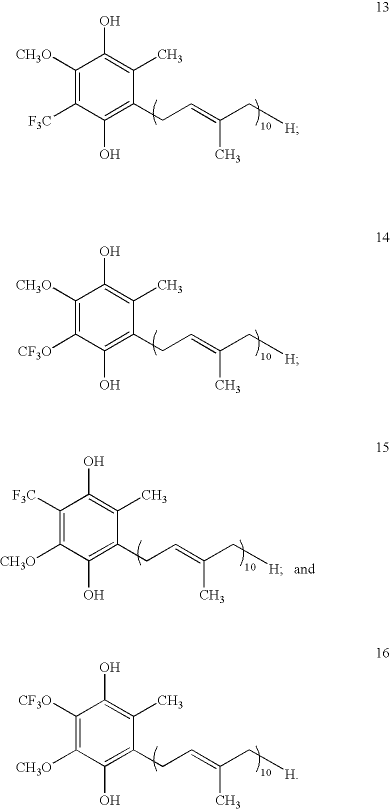

The hydrophilic moiety of the solubilizing agent is a hydrophilic molecule having a functional group, which can be used to chemically attach the hydrophilic molecule to the ubiquinol, either directly or through a linker moiety. Examples of said functional group include esterifiable hydroxy groups, carboxy groups, and amino groups. The hydrophilic molecule is preferably selected from the group consisting of polyalcohols, polyethers, polyanions, polycations, polyphosphoric acids, polyamines, polysaccharides, polyhydroxy compounds, polylysines, and derivatives thereof. Of those, polyethers are preferred, polyalkylene glycols being particularly preferred. The term “polyalkylene glycol” includes polymers of lower alkylene oxides, in particular polymers of ethylene oxide (polyethylene glycols) and propylene oxide (polypropylene glycols), having an esterifiable hydroxy group at least at one end of the polymer molecule, as well as derivatives of such polymers having esterifiable carboxy groups. The residue of the hydrophilic moiety is the entire hydrophilic molecule, except for the atom involved in forming the bond to the ubiquinol moiety or the linker moiety (i.e. an esterified hydroxy group, the oxygen molecule of an ether bond, a carboxy or amino group) or groups, such as terminal hydroxy groups of a polyethylene glycol molecule.

Polyethylene glycols are most particularly preferred for the practice of the present invention. Suitable polyethylene glycols may have a free hydroxy group at each end of the polymer molecule, or may have one hydroxy group etherified with a lower alkyl, e.g., a methyl group. Also suitable for the practice of the invention are derivatives of polyethylene glycols having esterifiable carboxy groups or amino groups, which may be used to form an amide bond. Polyethylene glycols are commercially available under the trade name PEG, usually as mixtures of polymers characterized by an average molecular weight. Polyethylene glycols having an average molecular weight from about 300 to about 5000 are preferred, those having an average molecular weight from about 600 to about 1000 being particularly preferred.

The linker moieties L1 and L2 can serve to connect the ubiquinol to the hydrophilic moiety. In an exemplary embodiment L1 and L2 are members independently selected from the following formulae: 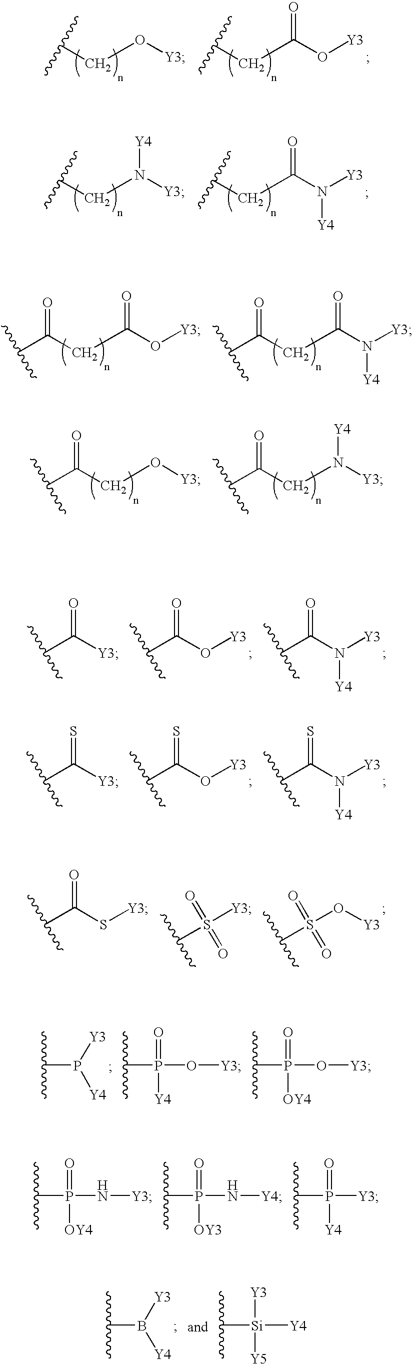
wherein the integer n is selected from 0 to 18. Y3 is a member selected from Y1 and Y2, and Y4 and Y5 are members independently selected from H, substituted or unsubstituted alkyl, substituted or unsubstituted heteroalkyl, substituted or unsubstituted aryl, substituted or unsubstituted heteroaryl and substituted or unsubstituted cycloalkyl and substituted or unsubstituted heterocycloalkyl.

Preferred linkers include diesters derived from an alkanedioic acid of the general formula HOOC—(CH₂)ₙ—COOH. For the practice of the present invention, alkanedioic acids with n from 0 to 18 are preferred, those with n from 6 to 10 being particularly preferred. Sebacic acid (n=8) is most particularly preferred.

Other preferred linkers include diethers derived from a substituted alkane. In ean exemplary embodiment the substituted alkane has the general structure X—(CH₂)ₙ—X′ wherein X and X′ independently represent a leaving group such as a halogen atom or a tosylate group. For the practice of the present invention, substituted alkanes with n from 0 to 18 are preferred, those with n from 6 to 10 being particularly preferred. The ether derived from a 1,10-substituted decane (n=10), such as 1,10-dibromodecane is most particularly preferred.

In an exemplary embodiment one or both of the phenolic hydroxy groups of the ubiquinol analog are derivatized with the hydrophilic moiety or the linker moiety. Exemplary solubilizing agents have the formula:

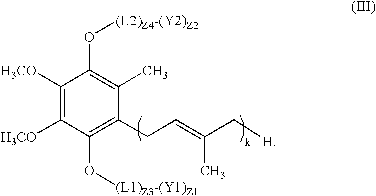

In another exemplary embodiment, one of the phenolic hydroxy groups of the ubiquinol analog is derivatized with a hydrophilic moiety of the invention. Exemplary solubilizing agents have the structure: 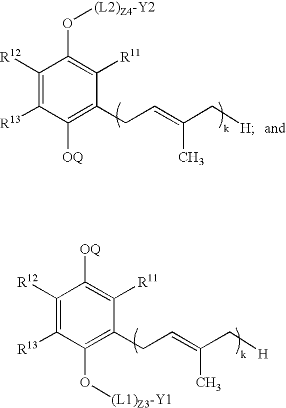
wherein Q is a member selected from H, a negative charge and a salt counter ion.

In an exemplary embodiment, one or both of the phenolic hydroxy groups of ubiquinol are esterified with the hydrophilic moiety or the linker moiety. Exemplary solubilizing agents have a formula which is a member selected from:

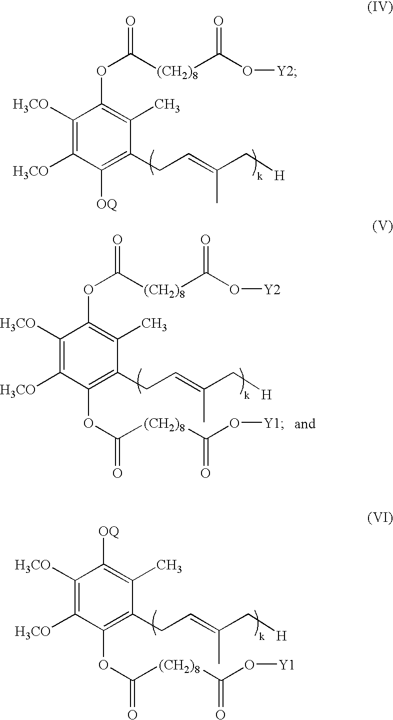

In another exemplary embodiment, one or both of the phenolic hydroxy groups of ubiquinol are part of an ether bond with the linker moiety. Exemplary solubilizing agents have a formula, which is a member selected from:

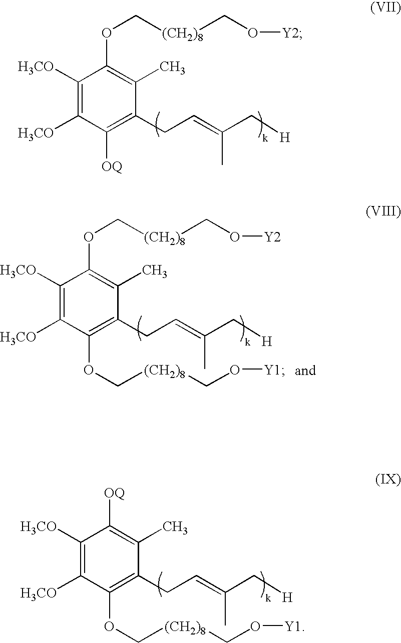

In another exemplary embodiment the invention provides a mixture including two or more solubilizing agents described herein. In an exemplary embodiment, the solubilizing agents are as described in Formula (I). In one example, the integer k may be constant, but some of the solubilizing agents include one hydrophilic moiety, while others include two hydrophilic moieties. In another embodiment, the mixture includes two regioisomers.

Hence, in one embodiment, the invention provides a mixture including a solubilizing agent of the invention, wherein Z1 and Z2 are each 1 and wherein the mixture further includes at least one member selected from: 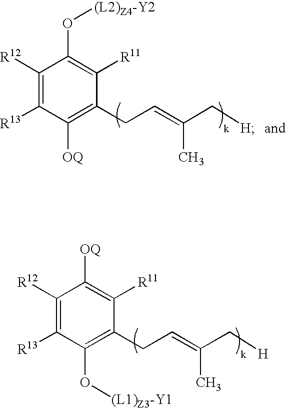
wherein Q is a member selected from H, a negative charge and a salt counter ion.

In another embodiment, the invention provides a mixture including a solubilizing agent of the invention, wherein Z2 is 1 and Z1 is 0, wherein the mixture further includes at least one member selected from: 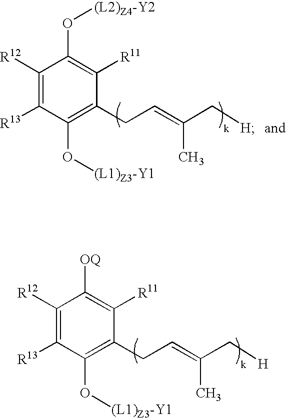
wherein Q is a member selected from H, a negative charge and a salt counter ion.

In yet another embodiment, the invention provides a mixture comprising a solubilizing agent of the invention, wherein Z1 is 1 and Z2 is 0, wherein the mixture further includes at least one member selected from: 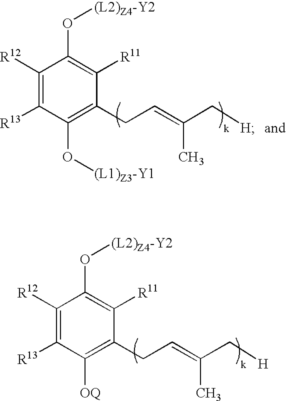
wherein Q is a member selected from H, a negative charge and a salt counter ion.

In an exemplary embodiment, the compounds in the mixture of solubilizing agents have structures according to Formulae (IV), (V), (VI), (VII), (VIII), and (IX).

According to another aspect, the present invention provides a water-soluble composition including the solubilizing agent according to Formula (I) and a bioactive lipophilic compound.

In a preferred embodiment, the compounds of the invention are water soluble. In one example, the compounds of the invention form micelles when added to an aqueous solution. The particle sizes of these micelles can be determined using art recognized methods, such as light scattering techniques. In one embodiment, the particle size is between about 300 nm and about 1 nm, preferably between about 200 nm and about 1 nm, more preferably between about 100 nm and about 1 nm. Smaller particle sizes are generally preferred.

Lipophilic compounds which can be solubilized using solubilizing agents of the present invention belong to a variety of therapeutic and chemical categories. In an exemplary embodiment the lipophilic compound is a bioactive, lipophilic compound, such as a pharmaceutical drug. In an exemplary embodiment the drug is a member selected from an antibiotic, in particular macrolide polyene antibiotics (e.g. amphotericin-B, nystatin, candicidin) and beta-lactam antibiotics, a tetracycline, an immunosuppressant (e.g., cyclosporine), an antitumor agent (e.g. paclitaxel and paclitaxel derivatives) and other poorly water-soluble drug molecules. In another exemplary embodiment the lipophilic compound is a member selected from a sterol, a vitamin (e.g., vitamin E), a provitamin, and a free radical scavenger (e.g., tocopherols). Various approaches to improve the aqueous solubility and bioavailability of these and other lipophilic compounds are known in the prior art, including formation of water-soluble complexes.

IIa. Pharmaceutical and Cosmetic Compositions

According to another aspect, the invention provides pharmaceutical or cosmetic formulations comprising a solubilizing agent described herein. In an exemplary embodiment, the solubilizing agent is according to Formula (I).

The solubilizing agents of the present invention can be easily incorporated into pharmaceutical or cosmetic formulations. In an exemplary embodiment, the lipophilic bioactive compound is then characterized by an improved bioavailability. Such formulations may further contain additional active ingredients and/or pharmaceutically or cosmetically acceptable additives or vehicles, including solvents, adjuvants, excipients, sweeteners, fillers, colorants, flavoring agents, lubricants, binders, moisturizing agents, preservatives and mixtures thereof. The formulations may be suitable for topical (e.g., a cream, lotion, gel, ointment, dermal adhesive patch), oral (e.g., a capsule, tablet, caplet, granulate), or parenteral (e.g., suppository, sterile solution) administration. Among the acceptable vehicles and solvents that may be employed for administration by injection are water, mildly acidified water, Ringer's solution and isotonic sodium chloride solution.

Lipophilic compounds, when combined with a solubilizing agent of the invention, may be administered to a warm-blooded animal, particularly a human, in need of the prophylaxis or therapy. The method comprises administering to such human or warm-blooded animal, an effective amount of a water-soluble composition including the solubilizing agent and the bioactive lipophilic compound. When the ubiquinol is linked to the hydrophilic moiety through a linker, which is cleavable in vivo, this composition provides an additional benefit for the patient. In vivo, the solubilizing agent is hydrolyzed by enzymes and is systemically converted back to the respective ubiquinol, which is further converted to the respective ubiquinone. One of these ubiquinones is Coenzyme Q₁₀ (CoQ₁₀). This natural compound has therapeutic potential for a number of disorders, including congestive heart failure, muscular dystrophy, periodontal disease, correction of drug-induced deficiencies, and immune restoration (AIDS, allergies). Coenzyme Q₁₀ is also of great interest to the cosmetic industry as an agent, which may slow down natural skin aging processes. In both clinical and cosmetic applications, preparations of coenzyme Q₁₀ with high bioavailability and solubility in aqueous media are usually desired. The dose of a bioactive lipophilic compound for treating the above-mentioned diseases or disorders vary upon the manner of administration, the age, sex, the body weight of the subject, and the condition being treated, and will ultimately be decided by the attending physician or veterinarian. Such an amount of the lipophilic bioactive compound as determined by the attending physician or veterinarian is referred to herein as a “therapeutically effective amount”.

IIb. Compositions for Organic Reactions

According to another aspect, the present invention provides a composition (e.g., a water-soluble composition) including a solubilizing agent as described herein and a lipophilic compound. In an exemplary embodiment the solubilizing agent is according to Formula (I). In an exemplary embodiment, the composition further includes a hydrophilic solvent, such as a water-based liquid. In an exemplary embodiment the lipophilic compound is a substrate of an organic reaction. Hence, the invention provides compositions including a solubilizing agent of the invention (for example according to Formula (I)), a lipophilic compound, which is a reactant in the organic reaction, a hydrophilic solvent, as well as one or more additional reactant. In an exemplary embodiment, each additional reactant is a members independently selected from catalysts, oxidation reagents, reducing agents, formylation reagents, halogenation reagents and the like. In another exemplary embodiment the hydrophilic solvent is a member selected from an alcohol and a water-based liquid (e.g., water). In another exemplary embodiment the alcohol is a member selected from methanol, ethanol, n-propanol, isopropanol and butanol.

III. The Methods

According to yet another aspect, the invention provides novel solubilizing agents described herein and methods of their preparation and purification. In an exemplary embodiment, the solubilizing agent has a structure according to Formula (I).

The ubiquinol analogs according to Formula (I), Formula (II), Formula (III) and their precursors of the corresponding ubiquinones are prepared by art-recognized methods or modifications thereof. Methods for the synthesis of quinone analogs are described by Lipshutz (Lipshutz et al., J. Am. Chem. Soc. 121: 11664-11673 (1999)), the disclosure of which is incorporated herein by reference. In addition, the synthesis of substituted methylene aromatic moieties, such as phenols, can be accomplished using methods described by U.S. Pat. No. 6,545,184 to Lipshutz et al., and U.S. Patent Application No. 20050148675 to Lipshutz et al.; the disclosures of which are also herein incorporated by reference.

Preferred linkers include diesters derived from an alkanedioic acid of the general formula HOOC—(CH₂)ₙ—COOH. For the practice of the present invention, alkanedioic acids with n from 0 to 18 are preferred, those with n from 6 to 10 being particularly preferred. Sebacic acid (n=8) is most particularly preferred. Those solubilizing agents can be prepared by reacting a hydrophilic compound of the general formula Y—OH with a spacer compound of the general formula Z—OC—(CH₂)ₙ—CO—Z, wherein Z is a member selected from OH, a leaving group, and further reacting the product so obtained with a ubiquinol analog. Halogens, in particular Cl and Br are preferred as the leaving group Z. In an exemplary embodiment the spacer moiety is activated through an NHS ester moiety.

Other preferred linkers include diethers derived from a substituted alkane. In an exemplary embodiment the substituted alkane has the general structure X—(CH₂)ₙ—X′ wherein X and X′ independently represent a leaving group such as a halogen atom or a tosylate group. In an exemplary embodiment the halogen is Br. The ether derived from a 1,10-substituted decane (n=10), such as 1,1 0-dibromodecane is particularly preferred.

Some solubilizing agents of Formula (I) may show a decreasing solubility in water with increasing temperature of the solution, which provides a convenient method of purifying these compounds.

According to another aspect, the present invention provides a method of solubilizing a bioactive lipophilic compound in a hydrophilic solvent, comprising contacting said bioactive lipophilic compound and said hydrophilic solvent in the presence of a solubilizing agent according to Formula (I). In an exemplary embodiment, the hydrophilic solvent (e.g., a water-based liquid, such as water) includes the solubilizing agent according to Formula (I).

According to yet another aspect, the present invention provides a method of performing a reaction between a lipophilic compound (e.g., a bioactive lipophilic compound) and a hydrophilic reactant, comprising contacting the hydrophilic reactant and a water-soluble composition of the invention, wherein the water-soluble composition includes a solubilizing agent according to Formula (I) and the lipophilic compound.

Various aspects of the present invention will be further illustrated by the following non-limiting examples.

### EXAMPLES

The following abbreviations are used throughout the Examples:

CoQ₁₀—coenzyme Q₁₀

Ub50—ubiquinol-50

PQS—polyoxyethanyl-ubiquinol-sebacate

A number following one of the above abbreviations indicates an average molecular weight of the polyoxyethanyl moiety (e.g., PEG) of the compound. For example, PQS-600 describes a compound of the invention derived from PEG-600, while PQS-1000 describes a compound of the invention derived from PEG-1000. A number followed by the abbreviation “Me” (e.g., PQS-750Me) indicates a polyoxyethanyl moiety capped with a methyl group (methoxypolyoxyethanyl).

### Example 1

### Preparation of Polyoxyethanyl-Ubiquinol Sebacate (POS-600 and POS-1000)

1.1. Preparation of PQS-600

To a solution of 0.83 g of Ub50 in 3 mL of dry toluene at 40° C., were added 1.33 mmole of triethylamine (TEA). A solution of 1.33 mmole of sebacoyl chloride in 2 mL of dry toluene was then added (dropwise, while stirring, and under anhydrous conditions) to the CoQ₁₀-TEA solution. The reaction was carried out for 10 min at room temperature, at which time 2 mmole of PEG-600 (polyethylene glycol, Sigma Chem. Co., product # P-3390) and 2.66 mmole of TEA dissolved in 3 mL of dry toluene were added dropwise to the reaction mixture. Stirring was continued for an additional 20 min at room temperature and the reaction mixture was extracted with saturated solution of NaCl (4×3 mL). The toluene was removed under reduced pressure leaving a waxy residue. The crude product was dissolved in 15 mL of water and water-insoluble materials were removed by filtration. The filtrate was lyophilized, yielding 0.8 g of pale-yellow waxy product (PQS-600). The product is water-soluble and in an aqueous solution forms micelles. The particle size for those micelles was found to be about 120 nm.

1.2. Preparation of PQS-1000

PQS-1000 was prepared from sebacoyl chloride and PEG-1000 according to the procedure outlined in Example 1.1. The product is water soluble in water and in an aqueous solution forms micelles. The particle size for those micelles was found to be about 80 nm. Smaller micelle size is generally preferred.

### Example 2

### Another Method for the Preparation of Polyoxvethanyl-Ubiqiuinol-Sebacates (e.g., POS-600)

A solution of 1 mmole of Ub50 and 1.33 mmole of TEA in 3 mL of dry toluene was added (dropwise, under anhydrous conditions, while stirring) to 1.33 mmole of sebacoyl chloride dissolved in 2 mL of dry toluene. The reaction was carried out for 10 min at room temperature, followed by a dropwise addition of 2 mmole of PEG-600 (polyethylene glycol, Sigma, P-3390) and 2.66 mmole of TEA dissolved in 3 mL of toluene. The reaction was continued for 20 min at room temperature with constant stirring. The reaction mixture was then extracted with saturated NaCl solution (4×3 mL) and toluene was evaporated under reduced pressure. The product was dissolved in 5 mL water and the residual toluene was further removed by co-evaporation with water under reduced pressure. The final waxy product was obtained by lyophilization.

PQS-1000 was obtained by linking polyethylene glycol (average molecular weight 1000, Sigma Chem. Co., product # P-3515) or methoxypolyethylene glycol (average molecular weight 750, Sigma Chem. Co., product # M-7018) to CoQ₁₀ using adipoyl, suberoyl, azelaoyl or dodecanedioyl dichlorides using the procedure described above for the preparation of PQS-600.

### Example 3

### Purification of Solubilizing Agents

A solubilizing agent prepared according to Examples 1 or 2 can be dissolved in water at 2:1 v/v ratio. The solution can be heated in a boiling water bath for approximately 2 min, until a visible precipitation occurred. This can then be followed by a brief centrifugation (at least 2000 X g) of the hot mixture to achieve separation of the precipitated product which is insoluble in hot water. The water phase (supernatant) can then be removed by decantation leaving a clear pellet of the product containing approximately 10% of water.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A solubilizing agent having a structure according to Formula (I): 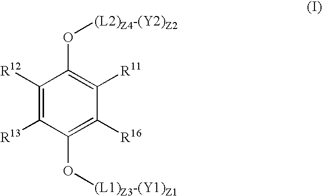
wherein
R¹¹, R¹² and R¹³ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl,
wherein
R¹² and R¹³, along with the atoms to which they are attached, are optionally joined to form a 4- to 8-membered ring;
R¹⁶ is a member selected from OR¹⁷, SR¹⁷, NR¹⁷R¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl
wherein
R¹⁷ and R¹⁸ are members independently selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl;
L1 and L2 are linker moieties, which are members independently selected from substituted or unsubstituted alkyl and substituted or unsubstituted heteroalkyl;
Y1 and Y2 are hydrophilic moieties, which are members independently selected from polyethers, polyalcohols and derivatives thereof,
Z1, Z2, Z3 and Z4 are members independently selected from 0 and 1, with the proviso that
(i) at least one of Z1 and Z2 is 1;
(ii) when Z4 and Z2 are both 0, (L2)_{z4}-(Y2)_{z2} is a member selected from H, a negative charge, and a salt counterion;
(iii) when Z3 and Z1 are both 0, (L1)_{z3}-(Y1)_{z1} is a member selected from H, a negative charge, and a salt counterion; and
with the further proviso that Y1, Y2, L1 and L2 do not comprise a labeling moiety, a targeting moiety or a drug moiety.

2. The solubilizing agent according to claim 1, wherein said linker moiety is a member selected from: 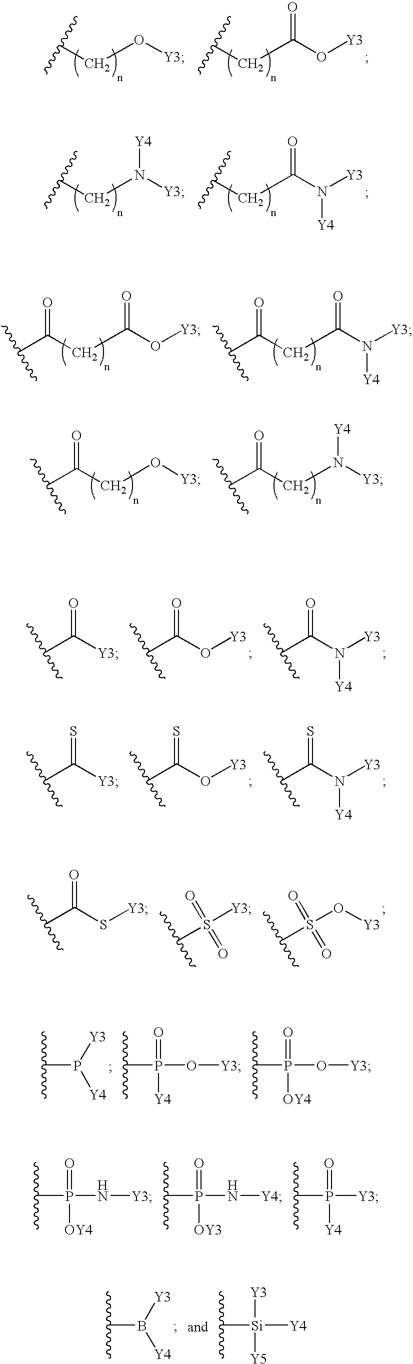
wherein
n is an integer selected from 0 to 18;
Y3 is a member selected from Y1 and Y2; and
Y4 and Y5 are members independently selected from H, substituted or unsubstituted alkyl, substituted or unsubstituted heteroalkyl, substituted or unsubstituted aryl, substituted or unsubstituted heteroaryl and substituted or unsubstituted cycloalkyl and substituted or unsubstituted heterocycloalkyl.

3. The solubilizing agent according to claim 1, having a structure according to Formula (II):

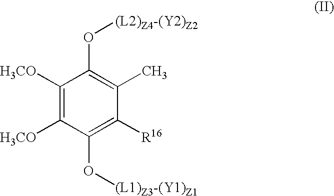

4. The solubilizing agent according to claim 2, wherein at least one of said Y1 and Y2 is a polyether.

5. The solubilizing agent according to claim 4, wherein said polyether is polyethylene glycol.

6. The solubilizing agent according to claim 5, wherein the polyethylene glycol has an average molecular weight of from about 300 to about 5000.

7. The solubilizing agent according to claim 6, wherein the polyethylene glycol has an average molecular weight of from about 600 to about 1000.

8. The solubilizing agent according to claim 1, wherein R¹⁶ comprises a structure according to Formula (III): 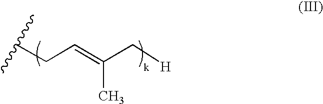
wherein
k is an integer selected from 1 to 13.

9. The solubilizing agent according to claim 8, wherein said k is 10.

10. A mixture comprising a solubilizing agent according to claim 1, wherein Z1 and Z2 are each 1, said mixture further comprising at least one member selected from: 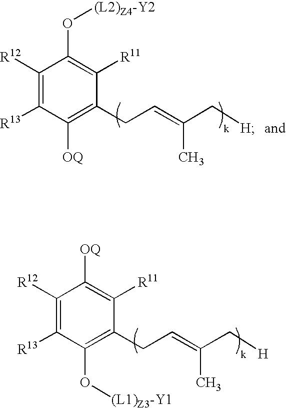
wherein Q is a member selected from H, a negative charge and a salt counter ion.

11. A mixture comprising a solubilizing agent according to claim 1, wherein Z2 is 1 and Z1 is 0, said mixture further comprising at least one member selected from: 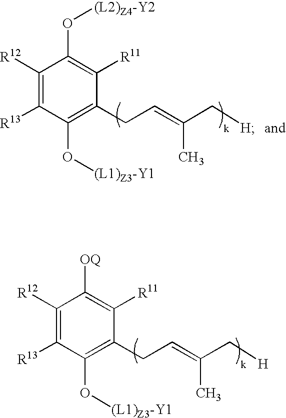
wherein Q is a member selected from H, a negative charge and a salt counter ion.

12. A mixture comprising a solubilizing agent according to claim 1, wherein Z1 is 1 and Z2 is 0, said mixture further comprising at least one member selected from: 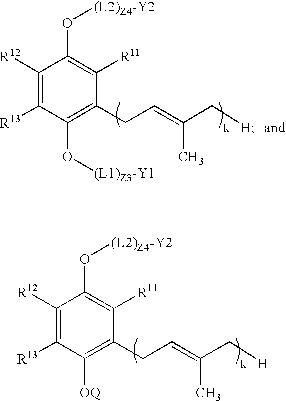
wherein Q is a member selected from H, a negative charge and a salt counter ion.

13. A water-soluble composition comprising:
a) a solubilizing agent according to claim 1; and
b) a lipophilic compound.

14. The water-soluble composition according to claim 13, wherein said lipophilic compound is a member selected from a pharmaceutical drug molecule, a sterol, a vitamin, a provitamin, an antibiotic, and a free radical scavenger.

15. The water-soluble composition according to claim 13, further comprising a pharmaceutically acceptable, water-soluble additive.

16. The water-soluble composition according to claim 15, wherein said additive is a member selected from a solvent, adjuvant, sweetener, filler, colorant, flavoring agent, lubricant, binder, moisturizing agent, preservative and mixtures thereof

17. A method of performing a reaction between a lipophilic compound and a hydrophilic reactant, said method comprising:
a) contacting said hydrophilic reactant and a water soluble composition according to claim 13,
thereby performing said reaction.

18. A method of solubilizing a lipophilic compound in a hydrophilic solvent, comprising:
a) contacting said bioactive lipophilic compound and said hydrophilic solvent,
wherein said hydrophilic solvent comprises a solubilizing agent of claim 1, thereby solubilizing said lipophilic compound.
